(19) United States
(12) Patent Application Publication
(10) Pub. No.: US 2008/0177156 A1
(43) Pub. Date: Jul. 24, 2008

(54) ISCHEMIA DETECTION USING PRESSURE SENSOR

(75) Inventors: Yi Zhang, Blaine, MN (US); Krzysztof Z. Siejko, Maple Grove, MN (US); Richard Fogoros, Pittsburg, PA (US); Abhilash Patangay, Little Canada, MN (US)

Correspondence Address: SCHWEGMAN, LUNDBERG & WOESSNER, P.A., P.O. BOX 2938, MINNEAPOLIS, MN, 55402, US

(73) Assignee: CARDIAC PACEMAKERS, INC., ST. PAUL, MN (US)

(21) Appl. No.: 11/624,974

(22) Filed: Jan. 19, 2007

(51) Int. Cl. A61B 5/0205; A61B 5/0215

(52) U.S. Cl. 600/301; 600/486

(57) ABSTRACT

This document discusses, among other things, a system and method for sensing a pulmonary artery pressure (“PAP”) signal of a pulmonary artery (“PA”) and computing an indication of a reduction of blood supply to at least a portion of a heart using information from the PAP signal. The reduction of blood supply to at least a portion of the heart can be detected using a PAP signal characteristic or measurement, using a change in the PAP, using an interval between multiple PAP signal features, using a mitral valve performance, or using information from the PAP and information from a different physiological signal, including a cardiac signal, a heart sound signal, right ventricular pressure signal, a left ventricular pressure signal, a blood pressure signal, and an oxygen saturation signal.

### TECHNICAL FIELD

This patent document pertains generally to cardiac health, and more particularly, but not by way of limitation, to ischemia detection using a pressure sensor.

### BACKGROUND

The heart is the center of the circulatory system of the human body. The left-sided chambers of the heart, including the left atrium and the left ventricle, draw blood from the lungs and pump it to the organs of the body to provide the organs with oxygenated blood. The right-sided chambers of the heart, including the right atrium and the right ventricle, draw blood from the organs and pump it into the lungs where the blood gets oxygenated. The organs of the body require oxygen to survive. Myocardial ischemia is generally the reduction of blood supply to the heart. Insufficient blood supply can cause the tissue of the heart to become hypoxic, or anoxic, and could eventually lead to the death of the heart tissue. Sudden occlusion of blood supply to the heart typically results in acute myocardial infarction, or a heart attack.

### Overview

This overview is intended to provide an overview of the subject matter of the present patent application. It is not intended to provide an exclusive or exhaustive explanation of the invention. The detailed description is included to provide further information about the subject matter of the present patent application.

Generally, left ventricle end diastolic pressure (“LVEDP”) is the pressure in the left ventricle (“LV”) at the end of the filling phase of the heart, and left atrial pressure (“LAP”) is the pressure in the left atrium (“LA”) throughout the cardiac cycle. Typically, LVEDP or LAP can be used to detect a reduction of blood supply to the heart or an occlusion of blood supply to the heart. Direct measurement of LVEDP or LAP generally requires direct placement of a catheter in the LV or the LA. Indirect measurement of LVEDP generally requires placement of a catheter in the pulmonary artery (“PA”) to measure the pulmonary capillary wedge pressure (“PCWP”), which is typically correlated to LVEDP. However, catheter placement is generally not preferred for chronic long-term monitoring. Thus, the present inventors have recognized, among other things, a need for detecting a reduction in blood supply to the heart using a chronic device.

This document discusses, among other things, a system and method for sensing a pulmonary artery pressure (“PAP”) signal of a PA and computing an indication of a reduction of blood supply to at least a portion of a heart using information from the PAP signal. The reduction of blood supply to at least a portion of the heart can be detected using a PAP signal characteristic or measurement, using a change in the PAP, using an interval between multiple PAP signal features, using a mitral valve performance, or using information from the PAP and information from a different physiological signal, including a cardiac signal, a heart sound signal, right ventricular pressure signal, a left ventricular pressure signal, a blood pressure signal, and an oxygen saturation signal.

In Example 1, a system includes an implantable chronic PA pressure sensor, configured to chronically sense a PAP signal of a PA, and an implantable or external processor, communicatively coupled to the PA pressure sensor to receive PAP information, wherein the processor is configured to use the PAP information to compute an indication of a reduction of blood supply to at least a portion of a heart.

In Example 2, the PA pressure sensor of Example 1 is optionally configured to be fixed to a location within the PA.

In Example 3, the processor of Examples 1-2 is optionally configured to compute the indication of a reduction of blood supply using a change in the PAP.

In Example 4, the processor of Examples 1-3 is optionally configured to detect at least one feature of the PAP signal, wherein the processor includes a time interval detector that is configured to detect at least one interval between the at least one feature of the PAP signal occurring at a first time and the at least one feature of the PAP signal occurring at a second time, and wherein the processor is configured to compute the indication of a reduction of blood supply to at least a portion of the heart using information from the at least one interval between the at least one feature of the PAP signal occurring at a first time and the at least one feature of the PAP signal occurring at a second time.

In Example 5, the processor of Examples 1-4 is optionally configured to compute the indication of a reduction of blood supply to at least a portion of the heart using at least one measurement correlative to at least one of a change in LV, a change in LV diastolic pressure, a change in LV volume, and a rate of pressure change in the LV (“LV dP/dt”).

In Example 6, the processor of Examples 1-5 is optionally configured to compute the indication of a reduction of blood supply to at least a portion of the heart using a detected change in a PA pressure characteristic, where the PA pressure characteristic includes at least one of a PA diastolic (“PAD”) pressure, a PA systolic (“PAS”) pressure, a mean PAP, and a rate of pressure change in the PA (“PA dP/dt”).

In Example 7, the processor of Examples 1-6 is optionally configured to compute the indication of a reduction of blood supply to a myocardium of a left ventricle.

In Example 8, the processor of Examples 1-7 is optionally configured to compute the indication of a reduction of blood supply to at least a portion of the heart by comparing at least a portion of the PAP information to a baseline.

In Example 9, the processor of Examples 1-8 is optionally configured to use the PAP information to detect an indication of mitral valve performance, and wherein the processor is configured to compute an indication of a reduction of blood supply to at least a portion of the heart using the detected indication of mitral valve performance.

In Example 10, the system of Examples 1-9 optionally includes an auxiliary physiological sensor, communicatively coupled to the processor, configured to sense a different physiological signal. The processor is also optionally configured to compute the indication of a reduction of blood supply to at least a portion of the heart using the PAP information and information from the different physiological signal.

In Example 11, the auxiliary physiological sensor of Examples 1-10 is optionally configured to sense a different physiological signal indicative of a reduction of blood supply to at least a portion of the heart.

In Example 12, the processor of Examples 1-11 is optionally configured to detect at least one feature of the different physiological signal and at least one feature of the PAP signal. The processor also optionally includes a time interval detector that is configured to detect at least one interval between the at least one different physiological signal feature and the at least one PAP signal feature. The processor is also optionally configured to compute the indication of a reduction of blood supply to at least a portion of the heart using the at least one interval between the at least one different physiological signal feature and the at least one PAP signal feature.

In Example 13, the auxiliary physiological sensor of Examples 1-12 optionally includes a cardiac sensor, configured to sense a cardiac signal as the different physiological signal.

In Example 14, the auxiliary physiological sensor of Examples 1-13 optionally includes a heart sound sensor, configured to sense a heart sound signal as the different physiological signal.

In Example 15, the auxiliary physiological sensor of Examples 1-14 optionally includes at least one of a right ventricular pressure sensor, a left ventricular pressure sensor, a blood pressure sensor, and an oxygen saturation sensor.

In Example 16, the processor of Examples 1-15 is optionally configured to compute the indication of a reduction of blood supply to at least a portion of the heart using the PAP signal, the different physiological signal, and at least one weighting factor for the PAP signal or the different physiological signal.

In Example 17, the at least one weighting factor for the PAP signal or the different physiological signal of Examples 1-16 optionally includes at least one of a signal-to-noise ratio (“SNR”) and a performance metric, wherein the performance metric includes at least one of a sensitivity, a specificity, a positive prediction value (“PPV”), and a negative prediction value (“NPV”).

In Example 18, the processor of Examples 1-17 is optionally configured to compute the indication of a reduction of blood supply to at least a portion of the heart using a temporal profile, wherein the temporal profile includes using the PAP information and information from the different physiological signal in a sequential manner.

In Example 19, the system of Examples 1-18 optionally includes an implantable respiration sensor, configured to sense a respiration signal, and an implantable or external respiration phase detector, coupled to the respiration sensor, configured to detect at least one phase of the respiration signal. The processor of Examples 1-18 is also optionally communicatively coupled to the respiration phase detector to receive respiration information. The processor is also optionally configured to use the PAP information and the respiration information to compute the indication of the reduction of blood supply to at least a portion of the heart, including at least one of the processor being configured to form a composite signal using at least a portion of the PAP signal over at least a portion of the at least one phase of the respiration signal, the processor being configured to obtain a gated PAP signal using information from the respiration phase detector, and the processor being configured to enable or disable the PA pressure sensor during at least a portion of at least one phase of the respiration signal.

In Example 20, a system includes means for chronically implantably sensing a pulmonary artery pressure (“PAP”) signal of a pulmonary artery (“PA”), such as by using a PA pressure sensor to sense the PAP signal of the PA. The system also includes means for using the PAP signal to compute an indication of a reduction of blood supply to at least a portion of a heart, such as by using a processor to detect a change in the PAP, using the processor to detect a deviation from a baseline, etc.

In Example 21, a method includes chronically implantably sensing a pulmonary artery pressure (“PAP”) signal of a pulmonary artery (“PA”). The method also includes using the PAP signal for computing an indication of a reduction of blood supply to at least a portion of a heart.

In Example 22, the sensing of Example 21 optionally includes using an implantable chronic PA pressure sensor configured to be fixed within the PA.

In Example 23, the using the PAP signal of Examples 21-22 optionally includes using a change in the PAP.

In Example 24, the method of Examples 21-23 optionally includes detecting at least one feature of the PAP signal. The method also optionally includes detecting at least one interval between the at least one feature of the PAP signal occurring at a first time and the at least one feature of the PAP signal occurring at a second time: The computing the indication of a reduction of blood supply to at least a portion of the heart of Examples 21-23 optionally includes using information from the at least one interval between the at least one feature of the PAP signal occurring at a first time and the at least one feature of the PAP signal occurring at a second time.

In Example 25, the computing the indication of a reduction of blood supply to at least a portion of the heart of Examples 21-24 optionally includes using at least one measurement correlative to at least one of a change in left ventricle (“LV”) pressure, a change in LV diastolic pressure, a change in LV volume, and a rate of pressure change in the LV (“LV dP/dt”).

In Example 26, the computing the indication of a reduction of blood supply to at least a portion of the heart of Examples 21-25 optionally includes using a detected change in a PA pressure characteristic, where the PA pressure characteristic includes at least one of a PA diastolic (“PAD”) pressure, a PA systolic (“PAS”) pressure, a mean PAP, and a rate of pressure change in the PA (“PA dP/dt”).

In Example 27, the computing the indication of a reduction of blood supply to at least a portion of the heart of Examples 21-26 optionally includes using at least one measurement of the PAP signal to compute an indication of a reduction of blood supply to a myocardium of a left ventricle.

In Example 28, the computing the indication of a reduction of blood supply to at least a portion of the heart of Examples 21-27 optionally includes comparing at least one measurement of the PAP signal to a baseline.

In Example 29, the method of Examples 21-28 optionally includes using the PAP signal for detecting an indication of mitral valve performance. The computing the indication of a reduction of blood supple to at least a portion of the heart of Examples 21-28 optionally includes using the detected indication of mitral valve performance.

In Example 30, the method of Examples 21-29 optionally includes sensing a different physiological signal, and computing the indication of a reduction of blood supply to at least a portion of the heart using the PAP signal and the different physiological signal.

In Example 31, the sensing a different physiological signal of Examples 21-30 optionally includes sensing a different physiological signal that is indicative of a reduction of blood supply to at least a portion of the heart.

In Example 32, the method of Examples 21-31 optionally includes detecting at least one feature of the different physiological signal, detecting at least one feature of the PAP signal, and detecting at least one interval between the at least one different physiological signal feature and the at least one PAP signal feature. The computing the indication of a reduction of blood supply to at least a portion of the heart optionally includes using the at least one interval between the at least one different physiological signal feature and the at least one PAP signal feature.

In Example 33, the sensing a different physiological signal of Examples 21-32 optionally includes sensing a cardiac signal as the different physiological signal.

In Example 34, the sensing the different physiological signal of Examples 21-33 optionally includes sensing a heart sound signal as the different physiological signal.

In Example 35, the sensing the different physiological signal of Examples 21-34 optionally includes sensing at least one of a right ventricular pressure signal, a left ventricular pressure signal, a blood pressure signal, and an oxygen saturation signal as the different physiological signal.

In Example 36, the computing the indication of a reduction of blood supply to at least a portion of the heart of Examples 21-35 optionally includes using the PAP signal, the different physiological signal, and at least one weighting factor for the PAP signal or the different physiological signal.

In Example 37, the using the at least one weighting factor for the PAP signal or the different physiological signal of Examples 21-36 optionally includes using at least of a signal-to-noise ratio (“SNR”) and a performance metric, wherein using the performance metric includes using at least one of a sensitivity, a specificity, a positive prediction value (“PPV”), and a negative prediction value (“NPV”).

In Example 38, the computing the indication of a reduction of blood supply to at least a portion of the heart of Examples 21-37 optionally includes using a temporal profile, wherein using the temporal profile includes using the PAP signal and the different physiological signal in a sequential manner.

In Example 39, the method of Examples 21-38 optionally includes sensing a respiration signal, detecting at least a portion of at least one phase of the respiration signal, and using the PAP signal and respiration signal information for computing the indication of the reduction of blood supply to at least a portion of the heart. The computing the indication of the reduction of blood supply to at least a portion of the heart of Examples 21-38 also optionally includes at least one of forming a composite signal using at least a portion of the PAP signal over at least a portion of at least one phase of the respiration signal, obtaining a gated PAP signal using information from the respiration signal, and enabling or disabling the sensing the PAP signal using at least a portion of at least one phase of the respiration signal.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates generally an example of a system including a PA pressure sensor and a processor.

FIG. 2 illustrates generally an example of a system including a heart, a PA pressure sensor, and a PA.

FIG. 3 illustrates generally an example of a system including a PA pressure sensor, a processor, and an auxiliary physiological sensor.

FIG. 4 illustrates generally an example of a system including a PA pressure sensor, a processor, and a cardiac sensor.

FIG. 5 illustrates generally an example of a system including a PA pressure sensor, a processor, and a heart sound sensor.

FIG. 6 illustrates generally an example of a system including a PA pressure sensor, a processor, a right ventricular pressure sensor, a left ventricular pressure sensor, a blood pressure sensor, and an oxygen saturation sensor.

FIG. 7 illustrates generally an example of a system including a PA pressure sensor, a processor, a respiration sensor, and a respiration phase detector.

FIG. 8 illustrates generally an example of a relationship between LVEDP and PA end-diastolic pressure (“PAEDP”), including a regression line.

FIG. 9 illustrates generally an example of LVEDP during balloon inflation and deflation and the rate of pressure change in the LV (“LV dP/dt”) during balloon inflation and deflation.

FIG. 10 illustrates generally an example of a method including sensing a PAP signal and computing an indication of a reduction of blood supply to at least a portion of a heart.

FIG. 11 illustrates generally an example of a method including sensing a PAP signal, detecting at least one feature of the PAP signal, detecting at least one interval between the at least one feature of the PAP signal occurring at a first time and the at least one feature of the PAP signal occurring at a second time, and computing an indication of a reduction of blood supply to at least a portion of a heart using the at least one interval.

FIG. 12 illustrates generally an example of a method including sensing a PAP signal, detecting an indication of mitral valve performance using the PAP signal, and computing an indication of a reduction of blood supply to at least a portion of a heart using the detected mitral valve performance.

FIG. 13 illustrates generally an example of a method including sensing a PAP signal, sensing a different physiological signal, and computing an indication of a reduction of blood supply to at least a portion of a heart using the PAP signal and the different physiological signal.

FIG. 14 illustrates generally an example of a method including sensing a PAP signal, detecting at least one feature of the PAP signal, sensing a different physiological signal, detecting at least one feature of the different physiological signal, detecting at least one interval between the at least one different physiological signal feature and the at least one PAP signal feature, and computing an indication of a reduction of blood supply to at least a portion of a heart using the at least one interval.

FIG. 15 illustrates generally an example of a method including sensing a PAP signal, sensing a respiration signal, detecting at least a portion of at least one phase of the respiration signal, forming a composite signal using at least a portion of the PAP signal over at least a portion of at least one phase of the respiration signal, obtaining a gated PAP signal using information from the respiration signal, enabling or disabling the sensing the PAP signal using at least a portion of at least one phase of the respiration signal, and computing an indication of a reduction of blood supply to at least a portion of a heart.

### DETAILED DESCRIPTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, logical and electrical changes may be made without departing from the scope of the present invention. The following detailed description is, therefore, not to be taken in a limiting sense, and the scope of the present invention is defined by the appended claims and their equivalents.

In this document, the terms “a” or “an” are used, as is common in patent documents, to include one or more than one. In this document, the term “or” is used to refer to a nonexclusive or, such that “A or B” includes “A but not B,” “B but not A,” and “A and B,” unless otherwise indicated. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

Generally, when at least 25% of the myocardium of the LV becomes acutely ischemic, the LVEDP and LV volume increase. Typically, in an acute coronary occlusion event, the LVEDP can increase by 10 mmHg and the rate of pressure change in the LV (“LV dP/dt”) can decrease by 500 mmHg/s in less than one minute.

During diastole, the PA diastolic pressure generally correlates to LVEDP. Thus, the change in LVEDP during a reduction of blood supply to the myocardium of the LV can typically be detected using the PA diastolic pressure. A reduction of blood supply, such as ischemia or myocardial infarction, to at least a portion of a heart, such as the myocardium of the LV, can generally be detected using information from an implantable PA pressure sensor. Using an implantable PA pressure sensor can allow for detection of a reduction of blood supply to at least a portion of the heart even before the subject undergoes symptoms of ischemia or myocardial infarction, or in asymptomatic subjects. Using an implantable PA pressure sensor can also typically allow for an enhanced detection ability by using the PA sensor information as well as other sensor information, such as a cardiac sensor information, a respiration sensor information, a right ventricle (“RV”) pressure sensor information, a LV pressure sensor information, a blood pressure sensor information, and an oxygen saturation sensor information, and separately can generally allow for trending capability. Further, using a non-lead-based PA pressure sensor can generally be advantageous for chronic monitoring of PA pressure.

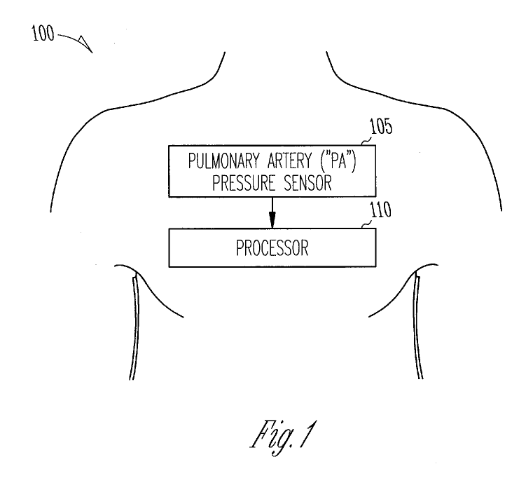

FIG. 1 illustrates generally an example of a system 100 including a PA pressure sensor 105 and a processor 110. The PA pressure sensor 105 generally includes an implantable PA pressure sensor, configured to be located in a PA of a subject. In certain examples, the processor 110 can be an implantable component, an external component, or a combination or permutation of an implantable component and an external component.

Generally, the PA pressure sensor 105 can be configured to sense a pulmonary artery pressure (“PAP”) signal of the PA of the subject. The PAP signal can include any signal indicative of the PAP of the PA of the subject. The PA pressure sensor 105 can be configured to produce a PAP signal, such as an electrical or optical PAP signal, that includes information about the PAP of the PA of the subject. The PA pressure sensor 105 can include a chronic PA pressure sensor, configured to remain in the PA for a continuing or extended period of time.

In the example of FIG. 1, the PA pressure sensor 105 can include a chronic non-lead-based pressure sensor. In certain examples, the non-lead-based pressure sensor can be implemented as a stand-alone device. The non-lead-based pressure sensor can be implemented with or without another implantable medical device, such as a pacemaker, defibrillator, or other implantable medical device, or the non-lead-based pressure sensor can be configured to be located at a close proximity to the left side of the heart, such as within the PA, to receive a physiologic signal at a close proximity to the left side of the heart.

In an example, the PA pressure sensor 105 can include a sensor, such as a pressure sensor, being implantable in a blood vessel supporting blood into or out of a cavity of a heart, such as a pulmonary artery, an example of which is disclosed in the Porat et al. U.S. Pat. No. 6,278,078 entitled “SYSTEM AND METHOD FOR MONITORING A PARAMETER ASSOCIATED WITH THE PERFORMANCE OF A HEART,” (herein “Porat et al. '078”) assigned to Remon Medical Technologies, Ltd. A PA pressure sensor can be used for monitoring a parameter associated with the performance of the heart, including collecting information pertaining to a pressure and a flow within the blood vessel supporting blood into or out of the cavity of the heart, such as the pulmonary artery. A separate sensor, such as a pressure sensor, can be used within the heart for collecting information pertaining to a pressure in a first cavity of the heart, such as a ventricle, and processing and interpreting information from both sensors to yield information pertaining to the heart performance of a subject.

In another example, the PA pressure sensor 105 can include an implantable pressure sensor placed in the PA to sense the PAP signal, such as that disclosed in the commonly assigned Stahmann U.S. patent application Ser. No. 11/249,624 entitled “METHOD AND APPARATUS FOR PULMONARY ARTERY PRESSURE SIGNAL ISOLATION,” which is hereby incorporated by reference in its entirety, including its disclosure of sensing the PAP signal using the implantable pressure sensor placed in the PA. In other examples, other pressure sensor configurations can be used to sense the PAP signal.

The PA pressure sensor 105 can be configured to communicate with one or more than one implantable medical device (“IMD”), such as the processor 110, a cardiac rhythm management device, an external medical device, or a combination or permutation of the one or more than one IMD, the processor 110, the cardiac rhythm management device, and the external medical device. Certain examples of such sensors, sensor configurations, and communication systems and methods are discussed in more detail in the Mazar et al. U.S. patent application Ser. No. 10/943,626 entitled “SYSTEMS AND METHODS FOR DERIVING RELATIVE PHYSIOLOGIC PARAMETERS;” the Von Arx et al. U.S. patent application Ser. No. 10/943,269 entitled “SYSTEMS AND METHODS FOR DERIVING RELATIVE PHYSIOLOGIC MEASUREMENTS USING AN EXTERNAL COMPUTING DEVICE;” the Von Arx et al. U.S. patent application Ser. No. 10/943,627 entitled “SYSTEMS AND METHODS FOR DERIVING RELATIVE PHYSIOLOGIC PARAMETERS USING A BACKEND COMPUTING SYSTEM;” and the Chavan et al. U.S. patent application Ser. No. 10/943,271 entitled “SYSTEMS AND METHODS FOR DERIVING RELATIVE PHYSIOLOGIC PARAMETERS USING AN IMPLANTED SENSOR DEVICE;” and the U.S. patent application Ser. No. 10/943,271 entitled “SYSTEMS AND METHODS FOR DERIVING RELATIVE PHYSIOLOGIC MEASUREMENTS USING AN IMPLANTED SENSOR DEVICE,” all assigned to Cardiac Pacemakers, Inc., all of which are incorporated herein by reference in their entirety, and which are collectively referred to as the “Physiologic Parameter Sensing Systems and Methods Patents” in this document.

In the example of FIG. 1, the processor 110 can be communicatively coupled to the PA pressure sensor 105. The processor 110 can be configured to receive information from the PA pressure sensor 105, such as discussed in more detail in the Physiologic Parameter Sensing Systems and Methods Patents. Generally, the processor 110 can be configured to compute an indication of a reduction of blood supply, such as ischemia or myocardial infarction, to at least a portion of a heart, such as the myocardium of the left ventricle or other portion of the heart. This may involve using information from the PA pressure sensor 105, such as by using at least one detected PA pressure characteristic or other information from the PA pressure sensor 105.

In an example, the processor 110 can be configured to detect at least one PA pressure characteristic, such as a PA diastolic pressure (“PAD”), a PA systolic pressure (“PAS”), a mean (or other central tendency) PAP, a PA end-diastolic pressure (“PAEDP”), a rate of pressure change in the PA (“PA dP/dt”), a PA pulse pressure (“PAPP”), or other PA pressure characteristic, using PAP information, such as the PAP signal, from the PA pressure sensor 105. The processor 110 can be configured to compute an indication of a reduction of blood supply to at least a portion of the heart using the at least one detected PA pressure characteristic, such as by using at least one detected feature of the at least one detected PA pressure characteristic. The at least one detected feature of the at least one detected PA pressure characteristic can include at least one of at least one detected amplitude, at least one detected magnitude, at least one detected peak, at least one detected valley, at least one detected value, at least one detected change, at least one detected increase, at least one detected decrease, and at least one detected rate of change in the at least one PA pressure characteristic. In another example, the processor 110 can be configured to compute an indication of a reduction of blood supply to at least a portion of the heart using a combination or permutation of the at least one detected PA pressure characteristics or features.

In another example, the processor 110 can be configured to detect at least one signal correlative to at least one LV pressure characteristic, such as a LV pressure, a LV diastolic pressure, a LV systolic pressure, a LVEDP, a mean (or other central tendency) LV pressure, a LV volume, a LV dP/dt, or other LV pressure characteristic, using PAP information from the PA pressure sensor 105. The processor 110 can be configured to compute an indication of a reduction of blood supply to at least a portion of the heart using the at least one signal correlative to at least one LV pressure characteristic, such as by using at least one detected feature of the at least one detected signal correlative to the at least one LV pressure characteristic. The at least one detected feature of the at least one detected signal correlative to the at least one LV pressure characteristic can include at least one of at least one detected amplitude, at least one detected magnitude, at least one detected peak, at least one detected valley, at least one detected value, at least one detected change, at least one detected increase, at least one detected decrease, and at least one detected rate of change in the at least one detected signal correlative to at least one LV pressure characteristic. In another example, the processor 110 can be configured to compute an indication of a reduction of blood supply to at least a portion of the heart using a combination or permutation of the at least one detected signal correlative to at least one LV pressure characteristic or the at least one detected feature of such signal.

In the example of FIG. 1, the processor 110 can include a time interval detector. In an example, the time interval detector can be configured to detect at least one time interval between at least a first feature of the PAP signal occurring at a first time and at least a second feature of the PAP signal occurring at a second time. In certain examples, the processor 110 can be configured to compute one or more than one output, such as by using at least one mathematical operation and one or more than one interval, such as computing the difference between more than one interval, computing an average of more than one interval, computing an average of more than one interval, or computing one or more than one other output using at least one mathematical operation and one or more than one interval. In an example, the processor 110 can be configured to compute the indication of a reduction of blood supply, such as ischemia or myocardial infarction, to at least a portion of the heart, such as the myocardium of the left ventricle or other portion of the heart, using information from the at least one interval between the at least one feature of the PAP signal occurring at a first time and the at least one feature of the PAP signal occurring at a second time.

In an example, the processor 110 can be configured to compute the indication of a reduction of blood supply to at least a portion of the heart by comparing at least a portion of the PAP information to a baseline. In certain examples, the baseline can include at least one of a population-based baseline, a subject-based baseline, an absolute baseline, and an adaptive baseline. In an example, the population-based baseline can include a condition-specific population-based baseline, such as a population-based baseline for subjects having hypotension, hypertension, heart failure, another condition, or one or more than one combination or permutation of one or more than one condition. In an example, the indication of a reduction of blood supply to at least a portion of the heart can include at least a portion of the PAP information, such as the PAP, the PAD, the PAS, the mean PAP, the PA dP/dt, etc., meeting, exceeding, or deviating from the baseline such as by a threshold amount. In another example, the indication of a reduction of blood supply to at least a portion of the heart can be computed using information from the difference between at least a portion of the PAP information and the baseline. In an example, the baseline can be established or reestablished using the processor 110.

In another example, the processor 110 can be configured to compute the indication of a reduction of blood supply to at least a portion of the heart by comparing at least a portion of the at least one measurement correlative to the at least one LV pressure characteristic to a baseline. In an example, the indication of a reduction of blood supply to at least a portion of the heart can include at least a portion of the at least one measurement correlative to the at least one LV pressure characteristic, such as the LV pressure, the LV diastolic pressure, the LVEDP, the LV systolic pressure, the mean LV pressure, the LV volume, the LV dP/dt, etc., meeting, exceeding, or deviating from the baseline. In another example, the indication of a reduction of blood supply to at least a portion of the heart can be computed using information from the difference between at least a portion of the at least one measurement correlative to the at least one LV pressure characteristic and the baseline.

In another example, the processor 110 can be configured to detect an indication of mitral valve performance using the PAP information. Generally, mitral valve performance can include any indicator of mitral valve function or dysfunction, such as mitral regurgitation (“MR”), mitral valve dysfunction, improper mitral valve seat or closure, an abnormal or backward pressure characteristic in a PAP signal or a LV pressure signal, etc. Typically, an indicator of ischemia, myocardial infarction, or other reduction in blood supply to the heart, can include mitral valve performance, such as MR or other mitral valve dysfunction. The processor 110 can be configured to compute an indication of a reduction of blood supply to at least a portion of the heart using the detected indication of mitral valve performance, such as MR.

In another example, the processor 110 can be configured to provide a notification of the computed indication of a reduction of blood supply to at least a portion of the heart to an external device, such as an external repeater, IMD, or other device capable of communicating with the processor 110. In certain examples, the external repeater, IMD, or other device can be configured to communicate, such as by an e-mail or other communication, to a user, such as a physician or other caregiver, or the subject.

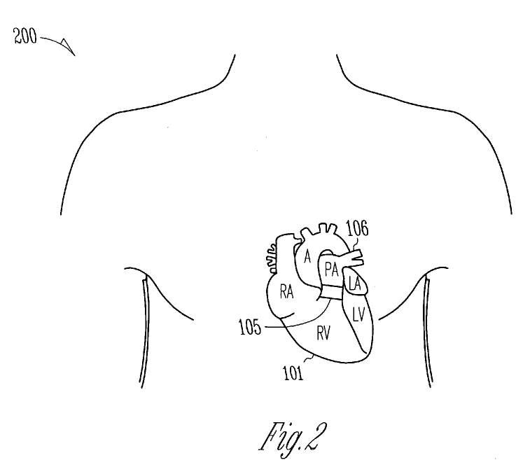

FIG. 2 illustrates generally an example of a system 200 including a heart 101, a PA pressure sensor 105, and a PA 106. Typically, the PA 106 of a subject carries blood from the RV of the heart 101 to the lungs. In the example of FIG. 2, the PA pressure sensor 105 is located in the PA 106 of the subject. The PA pressure sensor 105 can be fixed to a location within the PA 106, such as fixed to a wall of the PA 106. In an example, the PA pressure sensor 105 can be fixed to a location within the PA 106 without requiring a catheter, lead, or other component, to hold the PA pressure sensor 105 in the PA 106 of the subject.

In the example of FIG. 2, the PA pressure sensor 105 can be delivered, positioned, or anchored in a PA 106 of a subject, such as disclosed in the commonly assigned Chavan et al. U.S. patent application Ser. No. 11/216,738 entitled “DEVICES AND METHODS FOR POSITIONING AND ANCHORING IMPLANTABLE SENSOR DEVICES,” (herein “Chavan et al. '738”) which is hereby incorporated by reference in its entirety, including its disclosure of delivering, positioning, and anchoring a physiologic parameter sensor, such as a pressure sensor, to a bodily vessel, such as the pulmonary artery. In other examples, other methods of delivering, positioning, or anchoring physiologic parameter sensors can be used.

FIG. 3 illustrates generally an example of a system 300 including a PA pressure sensor 105, a processor 110, and an auxiliary physiological sensor 115. In certain examples, one or more of the PA pressure sensor 105, the processor 110, or the auxiliary physiological sensor 115, can be an implantable component, an external component, or a combination or permutation of an implantable component and an external component. For example, the processor 110 may be implantable, external, or distributed across both implantable and external locations.

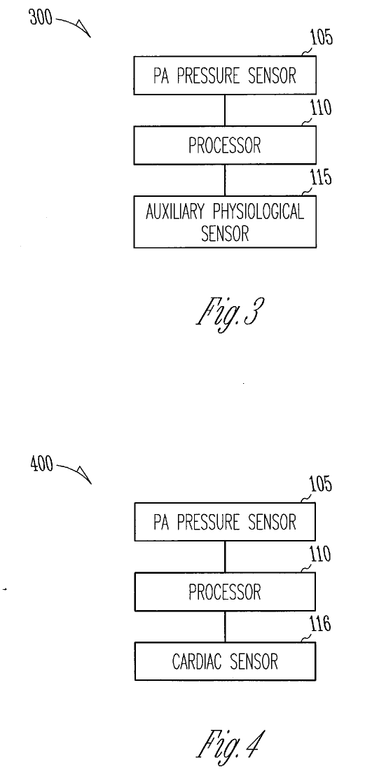

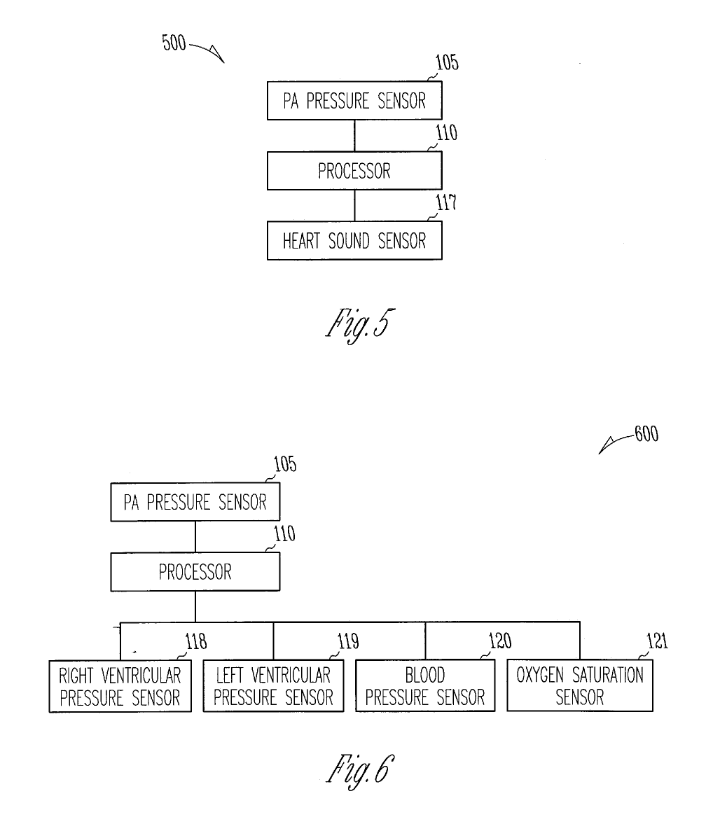

Generally, the auxiliary physiological sensor 115 can be configured to sense a different physiological signal of a subject, such as a physiological signal other than the PAP signal of the subject. The auxiliary physiological sensor 115 can include an implantable or external sensor configured to sense a different physiological signal of the subject, such as a cardiac sensor 116 (as shown in FIG. 4 below) configured to sense a cardiac signal of the subject, a heart sound sensor 117 (as shown in FIG. 5 below) configured to sense a heart sound signal of the subject, a right ventricular pressure sensor 118 (as shown in FIG. 6 below) configured to sense a right ventricular pressure signal of the subject, a left ventricular pressure sensor 119 (as shown in FIG. 6) configured to sense a left ventricular pressure signal of the subject, a blood pressure sensor 120 (as shown in FIG. 6) configured to sense a blood pressure signal of the subject, an oxygen saturation sensor 121 (as shown in FIG. 6) configured to sense an oxygen saturation signal of the subject, an impedance sensor to sense a cardiac impedance of the subject, an accelerometer, such as a lead based accelerometer, to sense an acceleration or deceleration of the subject, such as an acceleration or deceleration of the left ventricle of the subject, an activity sensor configured to sense an activity signal of the subject, a posture sensor configured to sense a posture of a subject, or other auxiliary physiological sensor configured to sense another physiological signal of the subject. Generally, the different physiological signal includes a physiological signal indicative of a reduction of blood supply to at least a portion of the heart, such as a cardiac signal, a heart sound signal, etc.

In this example, the processor 110 can be communicatively coupled to the auxiliary physiological sensor 115 and the PA pressure sensor 105. The processor 110 can be configured to receive information from the auxiliary physiological sensor 115, such as the different physiological signal, and information from the PA pressure sensor 105, such as the PAP signal. In an example, the processor 110 can be configured to compute an indication of a reduction of blood supply to at least a portion of the heart using information from the PA pressure sensor 105 and information from the auxiliary physiological sensor 115, such as the different physiological signal.

In the example of FIG. 3, the processor 1 10 can be configured to detect at least one feature of the different physiological signal, such as at least one of at least one detected amplitude, at least one detected magnitude, at least one detected peak, at least one detected valley, at least one detected value, at least one detected change, at least one detected increase, at least one detected decrease, and at least one detected rate of change in the different physiological signal.

The processor 110 can also include a time interval detector. In an example, the time interval detector can be configured to detect at least one interval between the at least one different physiological signal feature occurring at a first time and at least one PAP signal feature occurring at a second time. In certain examples, the processor 110 can be configured to compute one or more than one output using at least one mathematical operation and one or more than one interval, such as computing the difference between more than one interval, computing an average of more than one interval, computing a ratio of more than one interval, or computing other outcomes using at least one mathematical operation and one or more than one interval. In an example, the processor 110 can be configured to compute the indication of a reduction of blood supply to at least a portion of the heart using the at least one interval between the at least one different physiological signal feature occurring at the first time and the at least one PAP signal feature occurring at the second time.

FIG. 4 illustrates generally an example of a system 400 including a PA pressure sensor 105, a processor 110, and a cardiac sensor 116. In certain examples, the PA pressure sensor 105, the processor 110, or the cardiac sensor 116, can be an implantable component, an external component, or a combination or permutation of an implantable component and an external component, such as described above.

Generally, the cardiac sensor 116 can be configured to sense a cardiac signal of a subject. The cardiac signal can include any signal indicative of the electrical or mechanical cardiac activity of the heart, e.g., an electrocardiogram (“ECG”) signal, an impedance signal, an acceleration signal, etc. The cardiac sensor 116 can be configured to produce a cardiac signal, such as an electrical or optical cardiac signal, that includes information about the cardiac signal of the subject. The cardiac sensor 116 can include any device configured to sense the cardiac activity of the subject. In certain examples, the cardiac sensor 116 can include an intrinsic cardiac signal sensor, such as one or more than one electrode or lead to sense one or more than one depolarization, or a mechanical sensor, such as an impedance sensor or an accelerometer to sense one or more than one contraction.

In this example, the processor 110 can be communicatively coupled to the cardiac sensor 116 and the PA pressure sensor 105. The processor 110 can be configured to receive information from the cardiac sensor 116, such as the cardiac signal, and information from the PA pressure sensor 105, such as the PAP signal. In an example, the processor 110 can be configured to detect at least one feature of the cardiac signal. Typically, the at least one cardiac signal feature can include at least one feature or component of an ECG signal, e.g., at least one component of a P-wave, at least one component of a Q-wave, at least one component of a R-wave, at least one component of a S-wave, at least one component of a T-wave, or any combination or permutation of features or components of the ECG signal, or any mechanical cardiac features of a pressure signal or acceleration signal indicative of the cardiac activity of a subject.

In the example of FIG. 4, the processor 110 can be configured to compute an indication of a reduction of blood supply to at least a portion of the heart using information from the PA pressure sensor 105, such as the PAP signal, and information from the cardiac sensor 116, such as the cardiac signal, at least one detected feature of the cardiac signal, etc.

FIG. 5 illustrates generally an example of a system 500 including a PA pressure sensor 105, a processor 110, and a heart sound sensor 117. In certain examples, the PA pressure sensor 105, the processor 110, or the heart sound sensor 117, can be an implantable component, an external component, or a combination or permutation of an implantable component and an external component.

Generally, the heart sound sensor 117 can be configured to sense a heart sound signal of a subject. The heart sound signal can include any signal indicative of at least a portion of at least one heart sound of the subject. A heart sound of the subject can include an audible or mechanical noise or vibration indicative of blood flow through the heart or valve closures of the heart. The heart sound sensor 117 can be configured to produce a heart sound signal, such as an electrical or optical heart sound signal, that includes information about the heart sound signal of the subject. The heart sound sensor 117 can include any device configured to sense the heart sound signal of the subject. In certain examples, the heart sound sensor 117 can include an implantable sensor including at least one of an accelerometer, an acoustic sensor, a microphone, etc.

In an example, the heart sound sensor 117 can include an accelerometer configured to sense an acceleration signal indicative of the heart sound of the subject, such as that disclosed in the commonly assigned Carlson et al. U.S. Pat. No. 5,792,195 entitled “ACCELERATION SENSED SAFE UPPER RATE ENVELOPE FOR CALCULATING THE HEMODYNAMIC UPPER RATE LIMIT FOR A RATE ADAPTIVE CARDIAC RHYTHM MANAGEMENT DEVICE,” which is hereby incorporated by reference in its entirety, including its disclosure of accelerometer detection of heart sounds, or such as that disclosed in the commonly assigned Siejko et al. U.S. patent application Ser. No. 10/334,694, entitled “METHOD AND APPARATUS FOR MONITORING OF DIASTOLIC HEMODYNAMICS,” (herein “Siejko et al. '694”), which is hereby incorporated by reference in its entirety, including its disclosure of accelerometer detection of heart sounds. In other examples, other accelerometer or acceleration sensor configurations can be used to sense the heart sound signal.

In another example, the heart sound sensor 117 can include an acoustic sensor configured to sense an acoustic energy indicative of the heart sound of the subject, such as that disclosed in the commonly assigned Siejko et al. '694, incorporated by reference in its entirety, including its disclosure of acoustic sensing of heart sounds. In other examples, other acoustic sensor or microphone configurations can be used to sense the heart sound signal.

In this example, the processor 110 can be communicatively coupled to the heart sound sensor 117 and the PA pressure sensor 105. The processor 110 can be configured to receive information from the heart sound sensor 117, such as the heart sound signal, and information from the PA pressure sensor 105, such as the PAP signal. In an example, the processor 110 can be configured to detect at least one measurement, feature, characteristic, computation, or interval of at least a portion of at least one heart sound. In certain examples, this includes at least one of an amplitude of a heart sound, a magnitude of a heart sound, a total energy of a heart sound, an interval between one heart sound feature and another heart sound feature, at least one heart sound characteristic normalized by at least one other heart sound characteristic, etc. (e.g., an amplitude or magnitude of S1, an amplitude or magnitude of S2, an amplitude or magnitude of S3, an amplitude or magnitude of S4, the existence of a split-S2, a split-S2 time interval, a time interval between S1 and S2 (“S1-S2 time interval”), a time interval between S2 and S3 (“S2-S3 time interval”), a characteristic of S3 normalized by a characteristic of S1, etc.).

In the example of FIG. 5, the processor 110 can be configured to compute an indication of a reduction of blood supply to at least a portion of the heart using information from the PA pressure sensor 105, such as the PAP signal, and information from the heart sound sensor 117, such as the heart sound signal, at least one detected measurement, feature, characteristic, computation, or interval of at least a portion of at least one heart sound, etc.

In an example, an indication of a reduction of blood supply to at least a portion of the heart can be computed using a subsequent change in the heart sound signal from an established baseline heart sound signal, such as that disclosed in the commonly assigned Zhang et al. U.S. patent application Ser. No. 11/148,107 entitled “ISCHEMIA DETECTION USING HEART SOUND SENSOR,” which is hereby incorporated by reference in its entirety, including its disclosure of deeming that an ischemic event has occurred using a measured subsequent change in the heart sound signal from an established baseline heart sound signal.

FIG. 6 illustrates generally an example of a system 600 including a PA pressure sensor 105, a processor 110, a right ventricular pressure sensor 118, a left ventricular pressure sensor 119, a blood pressure sensor 120, and an oxygen saturation sensor 121. In certain examples, the PA pressure sensor 105, the processor 110, the right ventricular pressure sensor 118, the left ventricular pressure sensor 119, the blood pressure sensor 120, or the oxygen saturation sensor 121, can be an implantable component, an external component, or a combination or permutation of an implantable component and an external component.

In an example, the right ventricular pressure sensor 118 can be configured to sense a right ventricular pressure signal of a RV of a subject. The right ventricular pressure signal can include any signal indicative of the right ventricular pressure of the RV of the subject. The right ventricular pressure sensor 118 can be configured to produce a right ventricular pressure signal, such as an electrical or optical right ventricular pressure signal, that includes information about the right ventricular pressure of the RV of the subject.

In certain examples, the right ventricular pressure sensor 118 can include an implantable sensor, such as an implantable solid-state pressure transducer disposed on a catheter or an electrical lead, such as that disclosed in the commonly assigned Ding et al. U.S. Pat. No. 6,280,389 entitled “PATIENT IDENTIFICATION FOR THE PACING THERAPY USING LV-RV PRESSURE LOOP,” (herein “Ding et al. '389”) which is hereby incorporated by reference in its entirety, including its disclosure of measuring right ventricular pressure using a solid-state pressure transducer disposed on a catheter or an electrical lead, or at least one pressure transducer at a distal end, such as that disclosed in the commonly assigned Salo et al. U.S. Pat. No. 6,666,826 entitled “METHOD AND APPARATUS FOR MEASURING LEFT VENTRICULAR PRESSURE,” (herein “Salo et al. '826”) which is hereby incorporated by reference in its entirety, including its disclosure of attaching a pressure transducer to a catheter that is disposed within an open lumen of a lead system. In other examples, other pressure sensor configurations can be used to sense the right ventricular pressure signal.

In this example, the processor 110 can be communicatively coupled to the right ventricular pressure sensor 118 and the PA pressure sensor 105. The processor 110 can be configured to receive information from the right ventricular pressure sensor 118, such as the right ventricular pressure signal, and information from the PA pressure sensor 105, such as the PAP signal. In an example, the processor 110 can be configured to compute an indication of a reduction of blood supply to at least a portion of the heart using information from the PA pressure sensor 105 and information from the right ventricular pressure sensor 118.

In an example, the left ventricular pressure sensor 119 can be configured to sense a left ventricular pressure signal of a LV of the subject. The left ventricular pressure signal can include any signal indicative of the left ventricular pressure of the LV of the subject. The left ventricular pressure sensor 119 can be configured to produce a left ventricular pressure signal, such as an electrical or optical left ventricular pressure signal, that includes information about the left ventricular pressure of the LV of the subject.

In certain examples, the left ventricular pressure sensor 119 can include an implantable solid-state pressure transducer disposed on a catheter or an electrical lead, such as that disclosed in the commonly assigned Ding et al. '389, incorporated by reference in its entirety, including its disclosure of measuring left ventricular pressure using a solid-state pressure transducer disposed on a catheter or an electrical lead, or at least one pressure transducer at a distal end, such as that disclosed in the commonly assigned Salo et al. '826, incorporated by reference in its entirety, including its disclosure of attaching a pressure transducer to a catheter that is disposed within an open lumen of a lead system. In other examples, other pressure sensor configurations can be used to sense the left ventricular pressure signal.

In this example, the processor 110 can be communicatively coupled to the left ventricular pressure sensor 119 and the PA pressure sensor 105. The processor 110 can be configured to receive information from the left ventricular pressure sensor 119, such as the left ventricular pressure signal, and information from the PA pressure sensor 105, such as the PAP signal. In an example, the processor 110 can be configured to compute an indication of a reduction of blood supply to at least a portion of the heart using information from the PA pressure sensor 105 and information from the left ventricular pressure sensor 119.

In an example, the blood pressure sensor 120 can be configured to sense a blood pressure signal of the subject. The blood pressure signal can include an arterial blood pressure signal, an aortic blood pressure, a specific blood pressure signal, such as a LVEDP signal, or other blood pressure signal. The blood pressure sensor 120 can be configured to produce a blood pressure signal, such as an electrical or optical blood pressure signal, that includes information about the blood pressure of the subject.

In this example, the processor 110 can be communicatively coupled to the blood pressure sensor 120 and the PA pressure sensor 105. The processor 110 can be configured to receive information from the blood pressure sensor 120, such as the blood pressure signal, and information from the PA pressure sensor 105, such as the PAP signal. In an example, the processor 110 can be configured to compute an indication of a reduction of blood supply to at least a portion of the heart using information from the PA pressure sensor 105 and information from the blood pressure sensor 120.

In an example, the oxygen saturation sensor 121 can be configured to sense an oxygen saturation signal of the subject. The oxygen saturation signal can include any signal indicative of the level of oxygen in the blood. The oxygen saturation sensor 121 can be configured to produce an oxygen saturation signal, such as an electrical or optical oxygen saturation signal, that includes information about the level of oxygen in the blood of the subject.

In this example, the processor 110 can be communicatively coupled to the oxygen saturation sensor 121 and the PA pressure sensor 105. The processor 110 can be configured to receive information from the oxygen saturation sensor 121, such as the oxygen saturation signal, and information from the PA pressure sensor 105, such as the PAP signal. Generally, a reduction in blood supply to at least a portion of the heart can result in a reduced oxygen level to the at least a portion of the heart. In certain examples, if the level of oxygen in the blood meets, exceeds, or deviates from an established baseline, such as by a threshold amount, an indication of a reduction of blood supply to at least a portion of the heart can be computed. In an example, the processor 110 can be configured to compute an indication of a reduction of blood supply to at least a portion of the heart using information from the PA pressure sensor 105 and information from the oxygen saturation sensor 121.

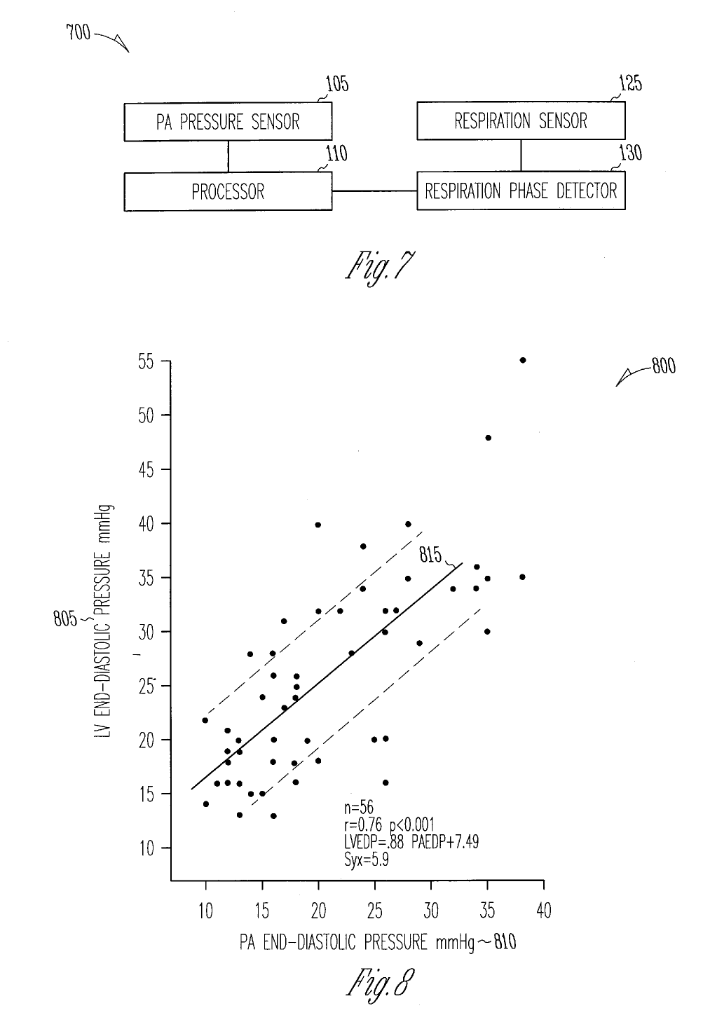

FIG. 7 illustrates generally an example of a system 700 including a PA pressure sensor 105, a processor 110, a respiration sensor 125, and a respiration phase detector 130. In certain examples, the PA pressure sensor 105, the processor 110, the respiration sensor 125, or the respiration phase detector 130, can be an implantable component, an external component, or a combination or permutation of an implantable component and an external component. In another example, some or all of the functionality of the respiration phase detector 130 can be implemented in the processor 110.

In this example, the respiration sensor 125 can be configured to sense a respiration signal of a subject. The respiration signal can include any signal indicative of the respiration of the subject, such as inspiration, expiration, or any combination, permutation, or component of the respiration of the subject. The respiration sensor 125 can be configured to produce a respiration signal, such as an electrical or optical respiration signal, that includes information about the respiration of the subject. In certain examples, the respiration sensor 125 can include an implantable sensor including at least one of an accelerometer, an impedance sensor, and a pressure sensor.

In an example, the respiration sensor 125 can include an accelerometer configured to sense an acceleration signal indicative of a cyclical variation indicative of respiration, such as that disclosed in the commonly assigned Kadhiresan et al. U.S. Pat. No. 5,974,340 entitled “APPARATUS AND METHOD FOR MONITORING REPSIRATORY FUNCTION IN HEART FAILURE PATIENTS TO DETERMINE EFFICACY OF THERAPY,” (herein “Kadhiresan et al. '340”) which is hereby incorporated by reference in its entirety, including its disclosure of using an accelerometer to detect respiration. In another example, the respiration sensor 125 can include a vibration sensor, such as that disclosed in the commonly assigned Hatlestad et al. U.S. Pat. No. 6,949,075 entitled “APPARATUS AND METHOD FOR DETECTING LUNG SOUNDS USING AN IMPLANTED DEVICE,” (herein “Hatlestad et al. '075”) which is hereby incorporated by reference in its entirety, including its disclosure of using a vibration sensor to detect respiration. In other examples, other accelerometer configurations can be used to sense the respiration signal.

In another example, the respiration sensor 125 can include an impedance sensor configured to sense an impedance signal indicative of respiration, such as that disclosed in the commonly assigned Kadhiresan et al. '340, incorporated by reference in its entirety. In another example, the respiration sensor 125 can include a transthoracic impedance sensor, such as that disclosed in the commonly assigned Hartley et al. U.S. Pat. No. 6,076,015 entitled “RATE ADAPTIVE CARDIAC RHYTHM MANAGEMENT DEVICE USING TRANSTHROACIC IMPEDANCE,” which is hereby incorporated by reference in its entirety, including its disclosure of using a thoracic impedance sensor to detect respiration. In other examples, other impedance sensor configurations can be used to sense the respiration signal.

In another example, the respiration sensor 125 can include a pressure sensor configured to sense a pressure signal indicative of respiration, such as that disclosed in the commonly assigned Hatlestad et al. '075, incorporated by reference in its entirety, including its disclosure of sensing a pressure signal indicative of respiration. In other examples, other pressure sensor configurations, such as a pulmonary artery pressure sensor, a ventricular pressure sensor, a thoracic pressure sensor, etc., can be used to sense a respiration signal.

In the example of FIG. 7, the respiration phase detector 130 can be coupled to the respiration sensor 125. The respiration phase detector 130 can be configured to receive the respiration signal from the respiration sensor 125. Generally, the respiration phase detector 130 can be configured to detect at least a particular portion of at least one phase of the respiration signal. In certain examples, this includes at least portion of at least one of an inspiration, an expiration, a transition between inspiration and expiration, and a transition between expiration and inspiration.

In this example, the processor 110 can be communicatively coupled to the respiration phase detector 130 and the PA pressure sensor 105. The processor 110 can be configured to receive information from the respiration phase detector 130, such as the at least a portion of at least one phase of the respiration signal, and information from the PA pressure sensor 105, such as the PAP signal.

In an example, the processor 110 can be configured to form a composite signal, such as an average or other signal, using information from the PA pressure sensor 105, such as at least a portion of the PAP signal, and information from the respiration phase detector 130, such as the at least a portion of the at least one phase of the respiration signal. The processor 110 can be configured to form an average signal using at least a portion of the PAP signal over at least a portion of the at least one phase of the respiration signal.

In another example, the processor 110 can be configured to obtain a gated PAP signal, such as by gating the PAP signal using information from the respiration phase detector 130. Typically, the processor 110 can gate the PAP signal using at least one respiration feature of the respiration signal to detect at least a portion of the PAP signal occurring during at least a portion of at least one phase of the respiration signal, such as the PAP signal during at least a portion of inspiration, expiration, the transition from inspiration to expiration or expiration to inspiration, etc.

In another example, the processor 110 can be configured to enable or disable the PA pressure sensor 105 during at least a portion of at least one phase of the respiration signal using information from the respiration phase detector 130. In certain examples, the PA pressure sensor 105 can be enabled for a period of time using information from the respiration phase detector 130, such as being enabled for at least a portion of at least one respiration phase or cycle every ten respiration phases or cycles, being enabled for at least a portion of at least one respiration phase or cycle every one hundred respiration phases or cycles, etc., or such as being enabled for at least a portion of at least one respiration phase or cycle once or more than once per hour, day, week, etc. In other examples, the PA pressure sensor 105 can be enabled during a respiration event, such as an apnea event, an increased or decreased respiratory rate, etc.

In the example of FIG. 7, the processor 110 can be configured to compute an indication of a reduction of blood supply to at least a portion of the heart using information from the PA pressure sensor 105, such as the PAP signal, and information from the respiration phase detector 130, such as at least a portion of at least one phase of the respiration signal.

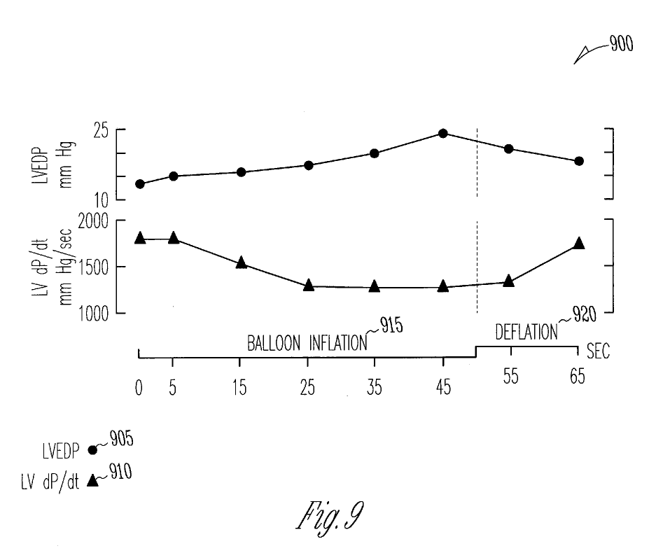

FIG. 8 illustrates generally an example 800 of a relationship between LV end-diastolic pressure (“LVEDP”) 805 and PA end-diastolic pressure (“PAEDP”) 810, including a regression line 815.

Regression analysis is generally used to determine the relationship between two or more measurements. A regression line of a set of data is typically the line of best fit, or the line that comes closest to all data points in the set. Correlation is generally the degree to which the two or more measurements are similar or related. A higher value of correlation corresponds to a higher degree of relation. Cleaner and more accurate signals typically have a higher value of correlation.

The present inventors have recognized that PAEDP generally correlates to LVEDP. In the example of FIG. 8, the regression line 815 is the line of best fit for the data of LVEDP 805 versus PAEDP 810. The correlation of the LVEDP versus PAEDP of example 800 is 0.76. Thus, PA pressure information can be used to detect a reduction of blood supply to at least a portion of the heart or an occlusion of blood supply to at least a portion the heart, such as ischemia, myocardial infarction, or other reduction or occlusion of blood supply to at least a portion of the heart.

FIG. 9 illustrates generally an example 900 of LVEDP 905 during a balloon inflation 915 and a balloon deflation 920 and the rate of pressure change in the LV (“LV dP/dt”) 910 during the balloon inflation 915 and the balloon deflation 920. Typically, balloon inflation 915 and balloon deflation 920 in a blood vessel, such as an artery or a vein, can be used to simulate a reduction of blood supply to a portion of the body. In the example 900, the reduction of blood supply is being simulated to the heart.

Generally, when the myocardium of the LV becomes acutely ischemic, the LVEDP 905 and the LV volume increase. In FIG. 9, during balloon inflation 915, LVEDP 905 increases roughly 10 mmHg. During this same period, LV dP/dt 910 decreases roughly 500 mmHg/s. Typically, as bodily activity increases, LVEDP increases. However, as bodily activity increases, LV dP/dt generally increases as well. Thus, by detecting LV dP/dt, it is possible to distinguish between a reduction of blood supply to at least a portion of the heart and an increase in LVEDP caused by activity.

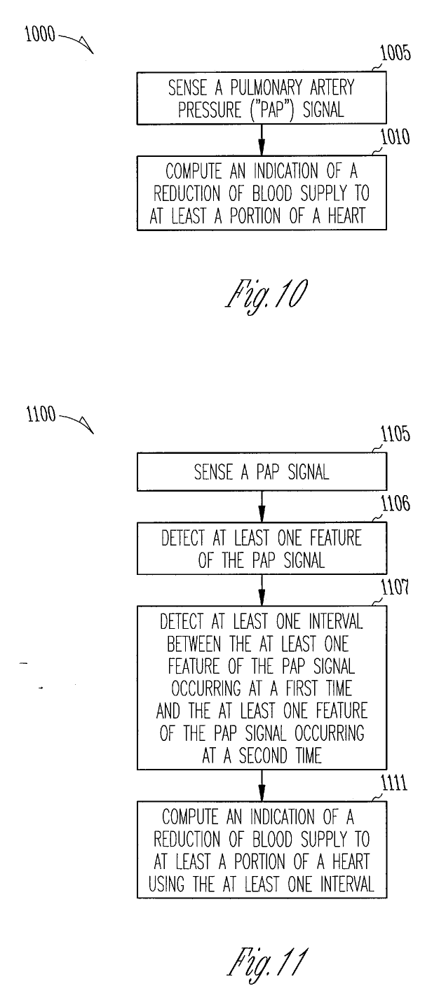

FIG. 10 illustrates generally an example of a method 1000 including sensing a pulmonary artery pressure (“PAP”) signal 1005 and computing an indication of a reduction of blood supply to at least a portion of a heart 1010.

At 1005, a PAP signal is sensed. The PAP signal can include any signal indicative of at least a portion of a PAP of a PA of a subject, such as a PAD, a PAS, a mean PAP, a PAEDP, a PA dP/dt, etc. In an example, the PAP signal can include a signal correlative to at least a portion of a LV pressure signal of the subject, such as a LV pressure, a LV diastolic pressure, a LV systolic pressure, a LVEDP, a mean LV pressure, a LV volume, a LV dP/dt, etc. In an example, the PAP signal can be sensed using the PA pressure sensor 105.

At 1010, a reduction of blood supply to at least a portion of the heart is computed. In an example, the reduction of blood supply to at least a portion of the heart can be computed using PAP information, such as at least a portion of the PAP signal, at least one PA pressure characteristic, at least one detected feature of the at least one PA pressure characteristic, etc. In certain examples, the reduction of blood supply to at least a portion of the heart can be computed using a detected change in PAD, such as an increase in PAD over a certain amount of time, a detected change in PA dP/dt, such as a decrease in PA dP/dt over a certain amount of time, or various combinations or permutations of detected changes in PAP information over certain amounts of time, such as synchronous or sequential intervals.

In an example, the reduction of blood supply to at least a portion of the heart can be computed by comparing at least a portion of the PAP information, such as the PAP, the PAD, the PAS, the mean PAP, the PA dP/dt, etc., to a baseline, such as a population-based baseline, a subject-based baseline, an absolute baseline, and an adaptive baseline. In an example, the population-based baseline can include a condition-specific population-based baseline, such as a population-based baseline for subjects having hypotension, hypertension, heart failure, other conditions, or one or more than one combination or permutation of one or more than one condition. In certain examples, the baseline can be established or reestablished using the processor 110 and at least a portion of the PAP information. In another example, the indication of a reduction of blood supply to at least a portion of the heart can be computed using information from the difference between at least a portion of the PAP information and the baseline.

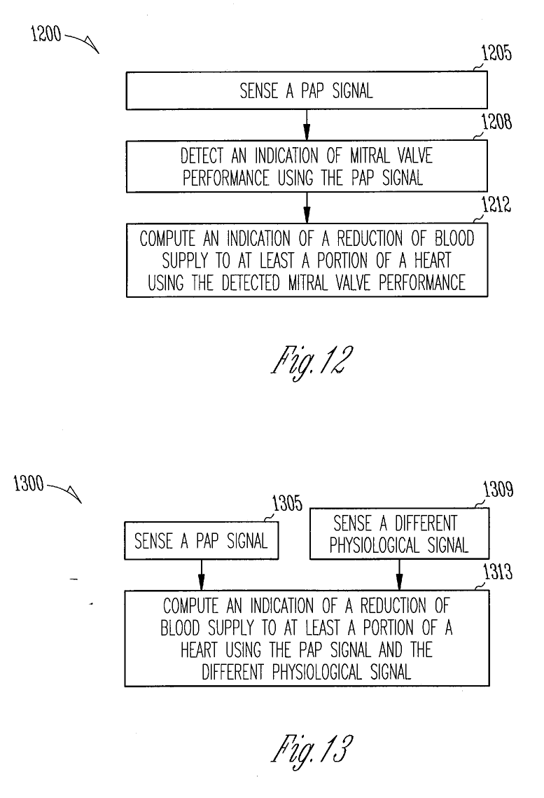

FIG. 11 illustrates generally an example of a method 1100 including sensing a PAP signal, detecting at least one feature of the PAP signal, detecting at least one interval between the at least one feature of the PAP signal occurring at a first time and the at least one feature of the PAP signal occurring at a second time, and computing an indication of a reduction of blood supply to at least a portion of a heart using the at least one interval.

At 1105, a PAP signal is sensed. The PAP signal can include any signal indicative of at least a portion of a PAP of a PA of a subject. In an example, the PAP signal can be sensed using the PA pressure sensor 105.

At 1106, at least one feature of the PAP signal is detected. The at least one feature of the PAP signal can include at least one of at least one detected amplitude, at least one detected magnitude, at least one detected peak, at least one detected valley, at least one detected value, at least one detected change, at least one detected increase, at least one detected decrease, and at least one detected rate of change in the at least one PA pressure characteristic. In an example, the at least one feature of the PAP signal can be detected using the processor 110.

At 1107, at least one interval between at least one feature of the PAP signal occurring at a first time and at least one feature of the PAP signal occurring at a second time is detected. In an example, the at least one interval can be detected using the processor 110 including a time interval detector.

At 1111, an indication of a reduction of blood supply to at least a portion of a heart can be computed using the at least one interval. In an example, a reduction of blood supply to at least a portion of the heart can be computed if the interval between a first feature of the PAP signal occurring at a first time, such as a detected PAD magnitude of a first level, and a second feature of the PAP signal occurring at a second time, such as a detected PAD magnitude of a second level, where the second level exceeds the first level by a certain amount, e.g., 50 mmHg, occurs within a certain amount of time, such as several seconds, e.g., 45 seconds. In another example, a 25% reduction of blood supply to at least a portion of the heart can be computed if the interval between a first feature of the PAP signal occurring at a first time, such as a detected LV dP/dt magnitude of a first level, and a second feature of the PAP signal occurring at a second time, such as a detected PAD magnitude of a second level, where the second level falls to a certain amount below the first level, e.g., 500 mmHg/sec, occurs within a certain amount of time, such as several seconds, e.g., 45 seconds.

FIG. 12 illustrates generally an example of a method 1200 including sensing a PAP signal, detecting an indication of mitral valve performance using the PAP signal, and computing an indication of a reduction of blood supply to at least a portion of a heart using the detected mitral valve performance.

At 1205, a PAP signal is sensed. The PAP signal can include any signal indicative of at least a portion of a PAP of a PA of a subject. In an example, the PAP signal can be sensed using the PA pressure sensor 105.

At 1208, an indication of mitral valve performance is detected using the PAP signal. Generally, mitral valve performance can include any indicator of mitral valve function or dysfunction, such as mitral regurgitation (“MR”), mitral valve dysfunction, improper mitral valve seat or closure, an abnormal or backward pressure characteristic in a PAP signal or a LV pressure signal, etc. The indication of mitral valve performance can be detected using the PAP information, such as the PAP signal, the signal correlative to at least a portion of a LV pressure signal, etc. In an example, the indication of mitral valve performance can be detected using abnormal pressure variations in the PAP signal indicative of an abnormal or backward pressure characteristic, or other mitral valve indicator.

At 1212, an indication of a reduction of blood supply to at least a portion of the heart is computed using the detected mitral valve performance. Typically, an indicator of ischemia, myocardial infarction, or other reduction in blood supply to the heart, can include mitral valve performance, such as MR or other mitral valve dysfunction. In an example, if the detected mitral valve performance indicates mitral valve dysfunction, MR, improper mitral valve seat or closure, an abnormal or backward pressure characteristic, etc., then an indication of a reduction of blood supply to at least a portion of the heart can be computed.

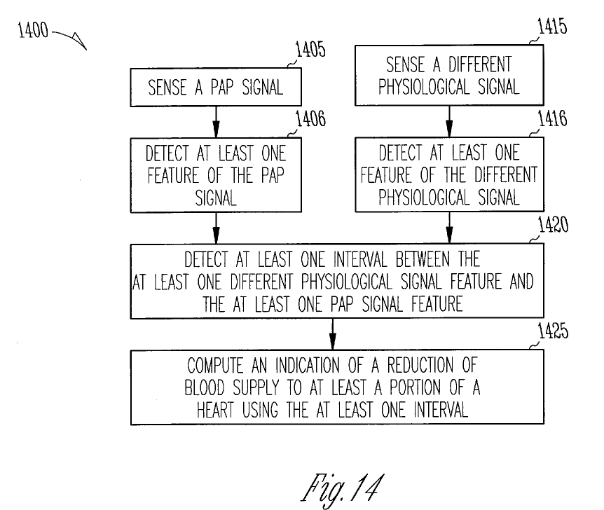

FIG. 13 illustrates generally an example of a method 1300 including sensing a PAP signal, sensing a different physiological signal, and computing an indication of a reduction of blood supply to at least a portion of a heart using the PAP signal and the different physiological signal.

At 1305, a PAP signal is sensed. The PAP signal can include any signal indicative of at least a portion of a PAP of a PA of a subject. In an example, the PAP signal can be sensed using the PA pressure sensor 105.

In an example, at 1305, the PAP signal is sensed continuously. In another example, the sensing the PAP signal can be triggered using information from the different physiological signal, such as heart-rate variability (“HRV”) information, heart-rate (“HR”) information, etc. In an example, if the HRV information or HR information deviates from an established baseline, the sensing the PAP signal can be triggered. Generally, triggering the sensing the PAP signal can reduce power consumption in the PA pressure sensor 105.

At 1309, a different physiological signal is sensed. The different physiological signal can include a physiological signal different than the PAP signal. In an example, the different physiological signal can include a physiological signal indicative of a reduction of blood supply to at least a portion of the heart, such as a cardiac signal, a heart sound signal, a right ventricular pressure signal, a left ventricular pressure signal, a blood pressure signal, an oxygen saturation signal, etc. In certain examples, the different physiological signal can be sensed using at least one of the auxiliary physiological sensor 115, the cardiac sensor 116, the heart sound sensor 117, the right ventricular pressure sensor 118, the left ventricular pressure sensor 119, the blood pressure sensor 120, the oxygen saturation sensor 121, or other physiological sensor capable of sensing a signal indicative of a reduction of blood supply to at least a portion of the heart.

In an example, at 1309, the different physiological signal can include at least a portion of the cardiac signal, such as the ST-segment of an ECG signal. Generally, the ST-segment of the ECG signal can be indicative of a reduction of blood supply to at least a portion of the heart. In an example, the ST-segment of the ECG signal can be detected using the cardiac sensor 116.

In another example, at 1309, the different physiological signal can include an impedance signal, such as a cardiac impedance signal. Generally, the cardiac impedance signal can be used to estimate a ventricular blood volume of the heart, and the ventricular blood volume of the heart can be indicative of a reduction of blood supply to at least a portion of the heart. In an example, the cardiac impedance signal can be detected using the impedance sensor.

In another example, at 1309, the different physiological signal can include an acceleration signal, such as a left ventricular acceleration signal. Generally, the acceleration signal can be indicative of a reduction of blood supply to at least a portion of the heart. In an example, the left ventricular acceleration signal can be detected using an accelerometer, such as a lead based accelerometer.

In other examples, the different physiological signal can include a physiological signal that is not indicative of a reduction of blood supply to at least a portion of the heart, such as an activity signal, a posture signal, or a respiration signal. In certain examples, the different physiological signals can be used to increase the sensitivity, sensitivity, or other function of one or more than one sensor, such as the PA pressure sensor 105. In certain examples, the different physiological signal that is not indicative of a reduction of blood supply to at least a portion of the heart can be detected using an activity sensor, a posture sensor, a respiration sensor, or other different physiological sensor.

At 1313, an indication of a reduction of blood supply to at least a portion of the heart can be computed using the PAP signal and the different physiological signal. Typically, using multiple sensors to sense the same or similar condition can increase the sensitivity or specificity of the sensing. In an example, the indication of a reduction of blood supply to at least a portion of the heart can be computed using the processor 110.

In an example, at 1313, an indication of a reduction of blood supply to at least a portion of the heart can be computed using the PAP signal and the cardiac signal, such as the ST-segment of the ECG signal. Typically, an elevated ST-segment, or an ST-segment that deviates from an established baseline, can be indicative of various physiological conditions ranging from an elevated heart rate, a change in position of the subject, ischemia, myocardial infarction, etc. Thus, by detecting an elevation in the ST-segment or a deviation from an established baseline ST-segment as well as using the PAP signal, an indication of a reduction in blood supply to at least a portion of the heart can be computed with an increased sensitivity or specificity.

In an example, at 1313, the indication of a reduction of blood supply to at least a portion of the heart can be computed using the PAP signal and the activity signal. Generally, the likelihood of ischemia, or other reduction of blood supply to at least a portion of the heart, is greater when the patient is active. In an example, the threshold, the baseline, etc., can be adapted to account for an increase in activity when computing the indication of a reduction of blood supply to at least a portion of the heart.

In an example, at 1313, the indication of a reduction of blood supply to at least a portion of the heart can be computed using the PAP signal, the different physiological signal, and at least one weighting factor for the PAP signal or the different physiological signal. Generally, different sensors have different detection sensitivity of specificity. Thus, an indication computed using a first signal may be a more accurate indication of a reduction of blood supply to at least a portion of the heart than by using a second signal. In certain examples, the at least one weighting factor can include at least one of a signal-to-noise ratio (“SNR”) and a performance metric, where a performance metric can include at least one of a signal sensitivity, a signal specificity, a positive prediction value (“PPV”), a negative prediction value (“NPV”), or other performance metrics.

In another example, at 1313, the indication of a reduction of blood supply to at least a portion of the heart can be computed using a temporal profile. In an example, the temporal profile can include using the PAP signal and the different physiological signal in a sequential manner, such as by detecting a first indication in a first signal, such as the PAP signal, and then detecting a second indication in a second signal, such as the different physiological signal.

In another example, at 1313, the indication of a reduction of blood supply to at least a portion of the heart can be computed by merging decisions from different sensors, such as by using fuzzy logic or other mathematical algorithm or operation.

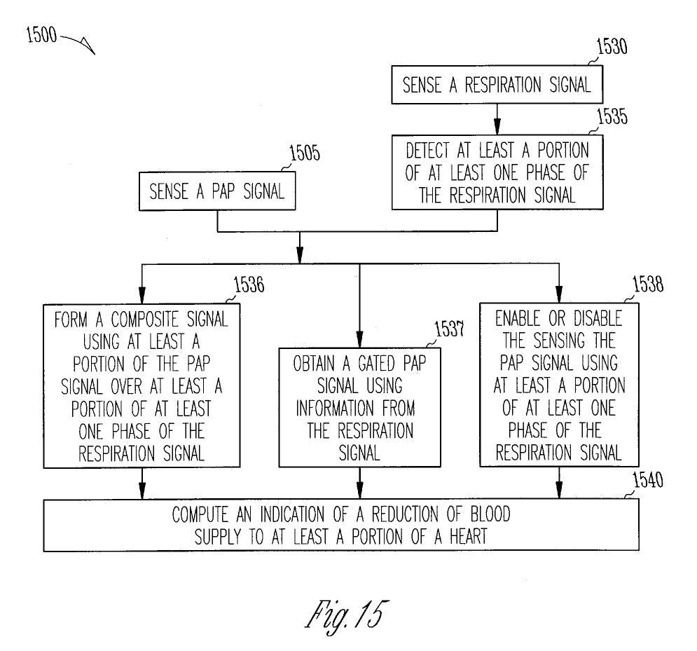

FIG. 14 illustrates generally an example of a method 1400 including sensing a PAP signal, detecting at least one feature of the PAP signal, sensing a different physiological signal, detecting at least one feature of the different physiological signal, detecting at least one interval between the at least one different physiological signal feature and the at least one PAP signal feature, and computing an indication of a reduction of blood supply to at least a portion of a heart using the at least one interval.

At 1405, a PAP signal is sensed. The PAP signal can include any signal indicative of at least a portion of a PAP of a PA of a subject. In an example, the PAP signal can be sensed using the PA pressure sensor 105.

At 1406, at least one feature of the PAP signal is detected. Generally, the at least one feature of the PAP signal can include any distinguishable feature of the PAP signal. The at least one feature of the PAP signal can include at least one of at least one detected amplitude, at least one detected magnitude, at least one detected peak, at least one detected valley, at least one detected value, at least one detected change, at least one detected increase, at least one detected decrease, and at least one detected rate of change in the PAP signal, such as the PAD, the PA dP/dt, the signal correlative to the LVEDP, etc.

At 1415, a different physiological signal is sensed. In certain examples, the different physiological signal can include at least one of a cardiac signal, a heart sound signal, a right ventricular pressure signal, a left ventricular pressure signal, a blood pressure signal, an oxygen saturation signal, or other physiological signal indicative of a reduction of blood supply to at least a portion of the heart.

In an example, at 1415, the different physiological signal includes the cardiac signal. The cardiac signal can include any signal indicative of the electrical or mechanical cardiac activity of a heart. In an example, the cardiac signal can be sensed using the cardiac sensor 116.

At 1416, at least one feature of the different physiological signal is detected. Generally, the at least one feature of the different physiological signal can include any distinguishable feature of the different physiological signal.

In an example, at 1416, the at least one feature of the different physiological signal includes at least one feature of the cardiac signal. The at least one feature of the cardiac signal can include at least one feature or component of an ECG signal, e.g., at least one component of a P-wave, at least one component of a Q-wave, at least one component of a R-wave, at least one component of a S-wave, at least one component of a T-wave, or any combination or permutation of features or components of the ECG signal, or any mechanical cardiac features of a pressure signal or acceleration signal indicative of the cardiac activity of a subject.

At 1420, at least one interval between the at least one different physiological signal feature and the at least one PAP signal feature is detected. In an example, the at least one interval can be detected using the processor 110.

At 1425, an indication of a reduction of blood supply to at least a portion of the heart is computed using the at least one interval between the at least one different physiological signal feature and the at least one PAP signal feature. Typically, the interval between physiological signal features can be indicative of a physiological event, e.g., the PAP of a subject generally rises slower during ischemia, increasing the interval between certain physiological markers and PAP signal features. In an example, an increasing interval between a cardiac signal feature, such as at least one component of the Q-wave or R-wave, and a PAP signal feature, such as at least one component of the PAD pressure, PAS pressure, etc., can be indicative of a reduction of blood supply to at least a portion of the heart.

FIG. 15 illustrates generally an example of a method 1500 including sensing a PAP signal, sensing a respiration signal, detecting at least a portion of at least one phase of the respiration signal, forming a composite signal using at least a portion of the PAP signal over at least a portion of at least one phase of the respiration signal, obtaining a gated PAP signal using information from the respiration signal, enabling or disabling the sensing the PAP signal using at least a portion of at least one phase of the respiration signal, and computing an indication of a reduction of blood supply to at least a portion of a heart.

At 1505, a PAP signal is sensed. The PAP signal can include any signal indicative of at least a portion of a PAP of a PA of a subject. In an example, the PAP signal can be sensed using the PA pressure sensor 105.

At 1530, a respiration signal is sensed. The respiration signal can include any signal indicative of the respiration of a subject, such as inspiration, expiration, or any combination, permutation, or component of the respiration of the subject. In an example, the respiration signal can be sensed using the respiration sensor 125.

At 1535, at least a portion of at least one phase of the respiration signal is detected. The at least one phase of the respiration signal can be detected using the respiration signal. In certain examples, the at least one phase of the respiration signal includes at least a portion of at least one of an inspiration, an expiration, a transition between inspiration and expiration, a transition between expiration and inspiration, etc. In certain examples, an inspiration, an expiration, the transitions between inspiration and expiration, etc., can be determined using the respiration signal, such as by differentiating the respiration signal to attain the slope of the respiration signal, by detecting peaks and valleys of the respiration signal, or by using other filtering methods or signal characteristics. In an example, the at least one phase of the respiration signal can be detected using the respiration phase detector 130.

In an example, at 1535, the at least one phase of the respiration signal includes an inspiration of one respiration cycle. A respiration cycle can include one full inspiration and expiration, one full expiration and inspiration, or any permutation or combination of a full inspiration and a full expiration. In other examples, the at least one phase of the respiration signal includes an expiration of one respiration cycle, a portion of an inspiration of one respiration cycle, a portion of an expiration of one respiration cycle, a portion of an inspiration and an expiration of one respiration cycle, a portion of an inspiration or an expiration of more than one respiration cycle, a portion of an inspiration and an expiration of more than one respiration cycle, etc.

At 1536, a composite signal is formed using at least a portion of the PAP signal over at least a portion of at least one phase of the respiration signal. Generally, forming a composite physiological signal over at least a portion of at least one phase of the respiration signal can remove noise or variation from the physiological signal due to respiration. In an example, the composite signal can include an average signal. In certain examples, the average signal can include an average PAS pressure signal over at least a portion of at least one phase of the respiration signal, such as inspiration, expiration, etc., an average PAD pressure signal over at least a portion of at least one phase of the respiration signal, etc.

At 1537, a gated PAP signal is obtained using information from the respiration signal. Typically, the PAP signal can be gated in order to detect at least a portion of the PAP signal, such as PAS during inspiration or expiration, PAD during inspiration or expiration, etc. In an example, the PAP signal can be gated using the processor 110.

In an example, at 1537, the PAP signal, or at least one characteristic or feature of-the PAP signal, can be gated during at least a portion of a first phase of the respiration signal, such as during at least a portion of inspiration, or at least a portion of expiration. In another example, the PAP signal, or at least one characteristic or feature of the PAP signal, can be gated using at least one feature of the respiration signal, such as the transition from inspiration to expiration, the transition from expiration to inspiration, etc., and a time interval, such as a time interval of 100-300 milliseconds, etc.

In other examples, a gated PAP signal can be obtained using information from the cardiac signal, such as the at least one feature or component of an ECG signal, or using information from the activity signal, such as when the subject is inactive.

At 1538, the sensing the PAP signal is enabled or disabled using at least a portion of at least one phase of the respiration signal. In an example, the PAP signal is sensed using the PA pressure sensor 105. Generally, enabling or disabling the PA pressure sensor can reduce power consumption. The sensing the PAP signal can be enabled or disabled during at least a portion of at least one phase of the respiration signal during at least one specific respiration cycle or time period, such as during inspiration every fifth respiration cycle, or during inspiration for ten consecutive respiration cycles once per hour, etc. In other examples, the sensing the PAP signal can be enabled during specific respiratory events, such as an apnea event, an increased or decreased respiratory rate, etc., or disabled during specific respiratory events, such as during normal respiratory function.

In other examples, the sensing the PAP signal can be enabled or disabled using information from the cardiac signal, such as enabling or disabling the sensing the PAP signal using the at least one feature of component of an ECG signal, or using information from the activity signal, such as enabling or disabling the sensing the PAP signal when the subject is inactive.

At 1540, an indication of a reduction of blood supply to at least a portion of the heart is computed using at least one of the composite signal, the gated PAP signal, and the enabled or disabled PAP signal. In an example, at 1540, the composite signal can be compared to a baseline, such as a baseline composite signal established using the processor 110 and the composite signal. In an example, if the composite signal deviates from the baseline, an indication of a reduction of blood supply to at least a portion of the heart can be computed. In another example, at 1540, the gated PAP signal can be compared to a baseline, such as a baseline gated PAP signal established or reestablished using the processor 1 10 and the gated PAP signal. In an example, if the gated PAP signal deviates from the baseline, an indication of a reduction of blood supply to at least a portion of the heart can be computed. In another example, at 1540, the enabled or disabled PAP signal can be compared to a baseline, such as a baseline enabled or disabled PAP signal established or reestablished using the processor 110 and the enabled or disabled PAP signal. In an example, if the enabled or disabled PAP signal deviates from the baseline, an indication of a reduction of blood supply to at least a portion of the heart can be computed.

In other examples, at 1540, other methods can be used to compute an indication of a reduction of blood supply to at least a portion of the heart using at least one of the composite signal, the gated PAP signal, and the enabled or disabled PAP signal, such as those disclosed in methods 1000-1400.

FIGS. 1-15 illustrate various examples, including sensing a pulmonary artery pressure (“PAP”) signal, detecting at least one feature of the PAP signal, detecting at least one interval between the at least one feature of the PAP signal occurring at a first time and the at least one feature of the PAP signal occurring at a second time, detecting an indication of mitral valve performance using the PAP signal, sensing a different physiological signal, sensing a cardiac signal, detecting at least one feature of the cardiac signal, detecting at least one interval between the at least one cardiac signal feature and the at least one PAP signal feature, sensing a respiration signal, detecting at least a portion of at least one phase of the respiration signal, forming a composite signal using at least a portion of the PAP signal over at least a portion of at least one phase of the respiration signal, obtaining a gated PAP signal using information from the respiration signal, enabling or disabling the sensing the PAP signal using at least a portion of at least one phase of the respiration signal, and computing an indication of a reduction of blood supply to at least a portion of a heart, etc., are disclosed. It is to be understood that these examples are not exclusive, and can be implemented either alone or in combination, or in various permutations or combinations.

It is to be understood that the above description is intended to be illustrative, and not restrictive. For example, the above-described embodiments (and/or aspects thereof) may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract is provided to comply with 37 C.F.R. §1.72(b), which requires that it allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment.

What is claimed is:

1. A system comprising:
an implantable chronic pulmonary artery (“PA”) pressure sensor, configured to chronically sense a pulmonary artery pressure (“PAP”) signal of a PA; and
an implantable or external processor, communicatively coupled to the PA pressure sensor to receive PAP information, wherein the processor is configured to use the PAP information to compute an indication of a reduction of blood supply to at least a portion of a heart.

2. The system of claim 1, wherein the PA pressure sensor is configured to be fixed to a location within the PA.

3. The system of claim 1, wherein the processor is configured to compute the indication of a reduction of blood supply using a change in the PAP.

4. The system of claim 1, wherein the processor is configured to detect at least one feature of the PAP signal;
wherein the processor includes a time interval detector that is configured to detect at least one interval between the at least one feature of the PAP signal occurring at a first time and the at least one feature of the PAP signal occurring at a second time; and
wherein the processor is configured to compute the indication of a reduction of blood supply to at least a portion of the heart using information from the at least one interval between the at least one feature of the PAP signal occurring at a first time and the at least one feature of the PAP signal occurring at a second time.

5. The system of claim 1, wherein the processor is configured to compute the indication of a reduction of blood supply to at least a portion of the heart using at least one measurement correlative to at least one of a change in left ventricle (“LV”) pressure, a change in LV diastolic pressure, a change in LV volume, and a rate of pressure change in the LV (“LV dP/dt”).

6. The system of claim 1, wherein the processor is configured to compute the indication of a reduction of blood supply to at least a portion of the heart using a detected change in a PA pressure characteristic, where the PA pressure characteristic includes at least one of a PA diastolic (“PAD”) pressure, a PA systolic (“PAS”) pressure, a mean PAP, and a rate of pressure change in the PA (“PA dP/dt”).

7. The system of claim 1, wherein the processor is configured to compute the indication of a reduction of blood supply to a myocardium of a left ventricle.

8. The system of claim 1, wherein the processor is configured to compute the indication of a reduction of blood supply to at least a portion of the heart by comparing at least a portion of the PAP information to a baseline.

9. The system of claim 1, wherein the processor is configured to use the PAP information to detect an indication of mitral valve performance, and wherein the processor is configured to compute an indication of a reduction of blood supply to at least a portion of the heart using the detected indication of mitral valve performance.

10. The system of claim 1, including:
an auxiliary physiological sensor, communicatively coupled to the processor, configured to sense a different physiological signal; and
wherein the processor is configured to compute the indication of a reduction of blood supply to at least a portion of the heart using the PAP information and information from the different physiological signal.

11. The system of claim 10, wherein the auxiliary physiological sensor is configured to sense a different physiological signal indicative of a reduction of blood supply to at least a portion of the heart.

12. The system of claim 10, wherein the processor is configured to detect at least one feature of the different physiological signal and at least one feature of the PAP signal;
wherein the processor includes a time interval detector that is configured to detect at least one interval between the at least one different physiological signal feature and the at least one PAP signal feature; and
wherein the processor is configured to compute the indication of a reduction of blood supply to at least a portion of the heart using the at least one interval between the at least one different physiological signal feature and the at least one PAP signal feature.

13. The system of claim 10, wherein the auxiliary physiological sensor includes a cardiac sensor, configured to sense a cardiac signal as the different physiological signal.

14. The system of claim 10, wherein the auxiliary physiological sensor includes a heart sound sensor, configured to sense a heart sound signal as the different physiological signal.

15. The system of claim 10, wherein the auxiliary physiological sensor includes at least one of a right ventricular pressure sensor, a left ventricular pressure sensor, a blood pressure sensor, and an oxygen saturation sensor.

16. The system of claim 10, wherein the processor is configured to compute the indication of a reduction of blood supply to at least a portion of the heart using the PAP signal, the different physiological signal, and at least one weighting factor for the PAP signal or the different physiological signal.

17. The system of claim 16, wherein the at least one weighting factor for the PAP signal or the different physiological signal includes at least one of a signal-to-noise ratio (“SNR”) and a performance metric, wherein the performance metric includes at least one of a sensitivity, a specificity, a positive prediction value (“PPV”), and a negative prediction value (“NPV”).

18. The system of claim 10, wherein the processor is configured to compute the indication of a reduction of blood supply to at least a portion of the heart using a temporal profile, wherein the temporal profile includes using the PAP information and information from the different physiological signal in a sequential manner.

19. The system of claim 1, including:
an implantable respiration sensor, configured to sense a respiration signal;
an implantable or external respiration phase detector, coupled to the respiration sensor, configured to detect at least one phase of the respiration signal; and
wherein the processor is communicatively coupled to the respiration phase detector to receive respiration information, and wherein the processor is configured to use the PAP information and the respiration information to compute the indication of the reduction of blood supply to at least a portion of the heart, including at least one of:
the processor being configured to form a composite signal using at least a portion of the PAP signal over at least a portion of the at least one phase of the respiration signal;
the processor being configured to obtain a gated PAP signal using information from the respiration phase detector; and
the processor being configured to enable or disable the PA pressure sensor during at least a portion of at least one phase of the respiration signal.

20. A system comprising:
means for chronically implantably sensing a pulmonary artery pressure (“PAP”) signal of a pulmonary artery (“PA”); and
means for using the PAP signal to compute an indication of a reduction of blood supply to at least a portion of a heart.

21. A method comprising:
chronically implantably sensing a pulmonary artery pressure (“PAP”) signal of a pulmonary artery (“PA”); and
using the PAP signal for computing an indication of a reduction of blood supply to at least a portion of a heart.

22. The method of claim 21, wherein the sensing includes using an implantable chronic PA pressure sensor configured to be fixed within the PA.

23. The method of claim 21, wherein the using the PAP signal includes using a change in the PAP.

24. The method of claim 21, including:
detecting at least one feature of the PAP signal;
detecting at least one interval between the at least one feature of the PAP signal occurring at a first time and the at least one feature of the PAP signal occurring at a second time; and
wherein computing the indication of a reduction of blood supply to at least a portion of the heart includes using information from the at least one interval between the at least one feature of the PAP signal occurring at a first time and the at least one feature of the PAP signal occurring at a second time.

25. The method of claim 21, wherein computing the indication of a reduction of blood supply to at least a portion of the heart includes using at least one measurement correlative to at least one of a change in left ventricle (“LV”) pressure, a change in LV diastolic pressure, a change in LV volume, and a rate of pressure change in the LV (“LV dP/dt”).

26. The method of claim 21, wherein computing the indication of a reduction of blood supply to at least a portion of the heart includes using a detected change in a PA pressure characteristic, where the PA pressure characteristic includes at least one of a PA diastolic (“PAD”) pressure, a PA systolic (“PAS”) pressure, a mean PAP, and a rate of pressure change in the PA (“PA dP/dt”).

27. The method of claim 21, wherein computing the indication of a reduction of blood supply to at least a portion of the heart includes using at least one measurement of the PAP signal to compute an indication of a reduction of blood supply to a myocardium of a left ventricle.

28. The method of claim 21, wherein computing the indication of a reduction of blood supply to at least a portion of the heart includes comparing at least one measurement of the PAP signal to a baseline.

29. The method of claim 21, including using the PAP signal for detecting an indication of mitral valve performance; and
wherein computing the indication of a reduction of blood supple to at least a portion of the heart includes using the detected indication of mitral valve performance.

30. The method of claim 21, including:
sensing a different physiological signal; and
computing the indication of a reduction of blood supply to at least a portion of the heart using the PAP signal and the different physiological signal.

31. The method of claim 30, wherein sensing a different physiological signal includes sensing a different physiological signal that is indicative of a reduction of blood supply to at least a portion of the heart.

32. The method of claim 30, including:
detecting at least one feature of the different physiological signal;
detecting at least one feature of the PAP signal;
detecting at least one interval between the at least one different physiological signal feature and the at least one PAP signal feature; and
wherein computing the indication of a reduction of blood supply to at least a portion of the heart includes using the at least one interval between the at least one different physiological signal feature and the at least one PAP signal feature.

33. The method of claim 30, wherein sensing a different physiological signal includes sensing a cardiac signal as the different physiological signal.

34. The method of claim 30, wherein sensing the different physiological signal includes sensing a heart sound signal as the different physiological signal.

35. The method of claim 30, wherein sensing the different physiological signal includes sensing at least one of a right ventricular pressure signal, a left ventricular pressure signal, a blood pressure signal, and an oxygen saturation signal as the different physiological signal.

36. The method of claim 30, wherein computing the indication of a reduction of blood supply to at least a portion of the heart includes using the PAP signal, the different physiological signal, and at least one weighting factor for the PAP signal or the different physiological signal.

37. The method of claim 36, wherein using the at least one weighting factor for the PAP signal or the different physiological signal includes using at least one of a signal-to-noise ratio (“SNR”) and a performance metric, wherein using the performance metric includes using at least one of a sensitivity, a specificity, a positive prediction value (“PPV”), and a negative prediction value (“NPV”).

38. The method of claim 30, wherein computing the indication of a reduction of blood supply to at least a portion of the heart includes using a temporal profile, wherein using the temporal profile includes using the PAP signal and the different physiological signal in a sequential manner.

39. The method of claim 21, including:
sensing a respiration signal;
detecting at least a portion of at least one phase of the respiration signal; and
using the PAP signal and respiration signal information for computing the indication of the reduction of blood supply to at least a portion of the heart, including at least one of:
forming a composite signal using at least a portion of the PAP signal over at least a portion of at least one phase of the respiration signal;
obtaining a gated PAP signal using information from the respiration signal; and
enabling or disabling the sensing the PAP signal using at least a portion of at least one phase of the respiration signal.
